(19) United States
(12) Patent Application Publication
(10) Pub. No.: US 2017/0086680 A1
(43) Pub. Date: Mar. 30, 2017

(54) APPARATUS FOR ACQUIRING OBJECT INFORMATION, INFORMATION PROCESSING METHOD, AND NON-TRANSITORY COMPUTER-READABLE STORAGE MEDIUM STORING PROGRAM

(71) Applicant: CANON KABUSHIKI KAISHA, Tokyo (JP)

(72) Inventors: Yoshihiro Hirata, Fuchu-shi (JP); Kazuhiko Fukutani, Yokohama-shi (JP)

(21) Appl. No.: 15/272,915

(22) Filed: Sep. 22, 2016

(30) Foreign Application Priority Data
Sep. 29, 2015 (JP) 2015-192212

(51) Int. Cl. A61B 5/00; A61B 5/145; G01S 15/89

(52) U.S. Cl. CPC A61B 5/0095; G01S 15/8965; A61B 5/14542; A61B 2576/00

(57) ABSTRACT

An apparatus for acquiring object information includes at least one receiving unit configured to receive an acoustic wave occurring in an object and output a signal, a liquid level acquiring unit configured to acquire information regarding a position of a liquid level of acoustic matching liquid provided between the object and the at least one receiving unit, and a processing unit configured to acquire object information by processing the signal by using the information regarding the position of the liquid level.

### BACKGROUND OF THE INVENTION

Field of Invention

The present invention generally relates to photoacoustic imaging, in particular it relates to an apparatus for acquiring object information based on acoustic waves from an object, an information processing method, and a non-transitory computer-readable storage medium storing a program.

Description of Related Art

Photoacoustic Imaging (PAI) is a technology for acquiring object information based on acoustic waves from an object. According to photoacoustic imaging principles, pulsed light is irradiated onto an object, and acoustic waves (photoacoustic waves) are generated from light absorbed within the object due to a photoacoustic effect. In this manner, the photoacoustic waves are used to image the inside of the object.

U.S. Patent Application Publication No. 2011/0306865, discloses a photoacoustic imaging apparatus including a transducer for receiving acoustic waves generated within an object. U.S. Patent Application Publication No. 2011/0306865, discloses a container configured to accumulate water between an object and a transducer for acoustic matching. According to the U.S. Patent Application Publication No. 2011/0306865, water functions as acoustic matching liquid attempting acoustic matching between an object and a transducer. In some cases, electric signals output from the transducer may vary in accordance with changes in position of the liquid level of the acoustic matching liquid within the container. This tends to deteriorate the accuracy of information regarding the object to be acquired based on the electric signals.

### SUMMARY OF THE INVENTION

Embodiments of the present invention are directed to an apparatus which can suppress deterioration and improve accuracy of information to be obtained from an object even in a case where the position of the level of the acoustic matching liquid changes.

An apparatus according to the present invention includes at least one receiving unit configured to receive an acoustic wave occurring in an object and output a signal, a liquid level acquiring unit configured to acquire information regarding a position of a liquid level of acoustic matching liquid provided between the object and the at least one receiving unit, and a processing unit configured to acquire object information by processing the signal by using the information regarding the position of the liquid level.

Further features of the present invention will become apparent from the following description of exemplary embodiments with reference to the attached drawings.

### BRIEF DESCRIPTION OF THE DRAWINGS

FIG. 1 is a schematic diagram illustrating a photoacoustic apparatus according to a first exemplary embodiment.

FIG. 2 illustrates a concrete example of a computer according to the first exemplary embodiment.

FIG. 3 is a flowchart for an object information acquiring method according to the first exemplary embodiment.

FIG. 4 is a dataflow of the object information acquiring method according to the first exemplary embodiment.

FIG. 5 illustrates a concrete example of a liquid level detecting unit according to the first exemplary embodiment.

FIG. 6 is a diagram illustrating the presence/absence of an acoustic matching according to the first exemplary embodiment.

FIG. 7 is a flowchart for an object information acquiring method according to a second exemplary embodiment.

### DESCRIPTION OF THE EMBODIMENTS

With reference to drawings, exemplary embodiments of the present invention will be described below.

### First Exemplary Embodiment

### System Configuration

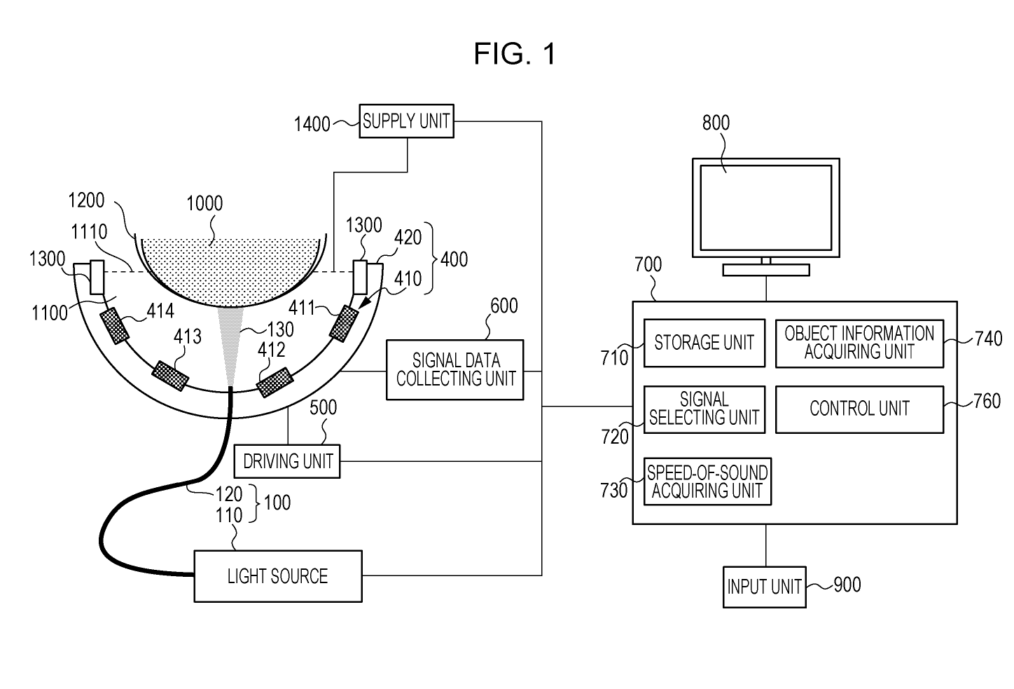

With reference to FIG. 1, a configuration of a photoacoustic apparatus functioning as an object information acquiring apparatus according to a first exemplary embodiment will be described. FIG. 1 is a schematic view illustrating a photoacoustic apparatus according to the first exemplary embodiment. The photoacoustic apparatus includes a light irradiating unit 100, an acoustic wave receiving unit 400 (hereinafter, called a receiving unit), a driving unit 500, a signal data collecting unit 600, a computer 700, a display unit 800, an input unit 900, a holding unit 1200, a liquid level detecting unit 1300, and a supply unit 1400. An object 1000 is a measurement target.

The light irradiating unit 100 irradiates pulsed light 130 to the object 1000 so that acoustic waves are generated within the object 1000. An acoustic wave generated by a photoacoustic effect due to light is called a photoacoustic wave. The receiving unit 400 is configured to output an electric signal as an analog signal, by receiving a photoacoustic wave and converting the photoacoustic wave into the electric signal. The signal data collecting unit 600 is configured to convert the electric signal being an analog signal output from the receiving unit 400 to a digital signal and output it to the computer 700. The computer 700 is configured to store the digital signal output from the signal data collecting unit 600 as signal data originating from a photoacoustic wave. The process from irradiation of light onto the object to output of digital signals to be stored as signal data will be called a “photoacoustic measurement”.

The computer 700 performs signal processing on the stored digital signals to generate information (object information) regarding the object 1000 and outputs it to the display unit 800. The display unit 800 is configured to display a numerical value and an image of the information regarding the object 1000. A doctor, as a user, can make a diagnosis by checking the numerical values and images of the information regarding the object displayed on the display unit 800.

The object information to be acquired by the photoacoustic apparatus according to this exemplary embodiment is at least one of information regarding a sound pressure at which photoacoustic waves are generated (initial sound pressure), an optical absorption energy density, an optical absorption coefficient, and a concentration of a substance contained in the object. The information regarding a concentration of a substance may include an oxyhemoglobin concentration, a deoxyhemoglobin concentration, a total hemoglobin concentration, an oxygen saturation, or a combination of these parameters. The total hemoglobin concentration refers to a sum of an oxyhemoglobin concentration and a deoxyhemoglobin concentration. The oxygen saturation refers to a proportion of oxyhemoglobin to the total hemoglobin. The photoacoustic apparatus according to this embodiment may acquire distribution information describing a value of the information at each position (in a two dimensional or three dimensional space) or a representative value (such as a mean value) of the information regarding an object as object information.

Acoustic matching liquid 1100 is filled between the receiving unit 400 and the holding unit 1200. However, due to fluctuations of the liquid level 1110 of the acoustic matching liquid 1100, there may be a moment when a part of the holding unit 1200 is not completely immersed in the acoustic matching liquid 1100, that is, a moment when the object 1000 and the receiving unit 400 are not acoustically matched. The electric signal output from the receiving unit 400 at that moment is highly possibly deteriorated as compared to the electric signal generated when they are acoustically matched. Particularly, when the receiving unit 400 is moved by the driving unit 500, the liquid level of the acoustic matching liquid 1100 in response thereto changes, which may possibly prevent the acoustic matching.

Accordingly, the computer 700 according to this exemplary embodiment acquires object information by using information regarding a position of the liquid level 1110 of the acoustic matching liquid 1100 acquired by the liquid level detecting unit 1300 as a liquid level acquiring unit, instead of an electric signal acquired when the acoustic matching is not achieved. Because the object information can be acquired without using a deteriorated electric signal, the deterioration of acquired object information can be prevented and accuracy of measurement can be improved.

In a photoacoustic apparatus, acquiring an electric signal from object information may require information regarding a propagation speed (speed of sound) of an acoustic wave within the acoustic matching liquid 1100. The speed of sound within the acoustic matching liquid 1100 varies in accordance with the pressures at the corresponding positions therein. Processing an electric signal without consideration of the variations may deteriorate the acquired object information. Accordingly, the computer 700 in this exemplary embodiment acquires a pressure distribution within the acoustic matching liquid 1100 by using information regarding positions of the liquid level 1110 of the acoustic matching liquid 1100 acquired by the liquid level detecting unit 1300. The computer 700 further acquires a speed of sound within the acoustic matching liquid 1100 by using the pressure distribution within the acoustic matching liquid 1100. Thus, a speed of sound within the acoustic matching liquid 1100 can be acquired with higher precision compared to a case without consideration of the pressure distribution within the acoustic matching liquid 1100. Then, the computer 700 can acquire object information with high accuracy by using the highly precisely acquired speed of sound. The computer 700, which receives the information regarding the positions of the liquid level 1110 of the acoustic matching liquid 1100, may be the liquid level acquiring unit.

Components of the photoacoustic apparatus according to this exemplary embodiment will be described below.

### Light Irradiating Unit 100

A light irradiating unit 100 includes a light source 110 configured to emit pulsed light 130, and an optical system 120 which directs the pulsed light 130 emitted from the light source 110 to the object 1000.

The light emitted by the light source 110 may have a pulse width equal to or larger than 1 ns and equal to or smaller than 100 ns. The light may have a wavelength in a range approximately from 400 nm to 1600 nm. For imaging of a blood vessel in vicinity of a body surface at high resolution, the light may have a wavelength (equal to or larger than 400 nm and equal to or smaller than 700 nm) which can be highly absorbed by a blood vessel. For imaging a deep part of a living body on the other hand, the light may have a wavelength (equal to or larger than 700 nm and equal to or smaller than 1100 nm) which can typically be absorbed less by a background tissue (such as water and fat) of a living body.

The light source 110 may be a laser or a light emitting diode. A light source providing a wavelength that can be converted may be used for a measurement employing light having a plurality of wavelengths. When light having a plurality of wavelengths is irradiated to an object, a plurality of light sources may be prepared which can generate light rays having different wavelengths from each other, and the light rays may be irradiated alternately from the light sources. The plurality of light sources if used is also collectively called a light source. Various lasers may be used such as a solid-state laser, a gas laser, a dye laser, and a semiconductor laser. A pulsed laser such as an Nd:YAG laser and an alexandrite laser may be used. A Ti:sa laser and OPO (Optical Parametric Oscillators) laser which uses Nd:YAG laser light as excited light may be used.

The optical system 120 may be an optical device such as a lens, a mirror, and an optical fiber. In a case where the object 1000 is the breast, for example, pulsed light with increased beam diameters may be irradiated. Therefore, a light emitting unit in the optical system 120 may include a diffusing plate configured to diffuse light. For a higher resolution in a photoacoustic microscope on the other hand, the light emitting unit in the optical system 120 may be a lens to focus and irradiate a beam.

The light irradiating unit 100 may not include the optical system 120, but pulsed light 130 may be irradiated from the light source 110 directly to the object 1000.

### Receiving Unit 400

The receiving unit 400 includes a receiving element group 410 having receiving elements 411, 412, 413 to 414 each configured to receive an acoustic wave and output an electric signal therefrom and a supporting member 420 configured to support the receiving element group 410.

The receiving elements 411, 412, 413 to 414 may contain a piezoelectric ceramic material typically such as PZT (lead zirconate titanate) or a high molecule piezoelectric film material typically such as PVDF (PolyVinylidene DiFluoride). Other elements than such piezoelectric elements may be used instead. For example, capacitive micro-machined ultrasonic transducers (CMUT) or transducers employing a Fabry-Perot interferometer may be used. Any kinds of transducer may be selected as the receiving elements if the transducers can output an electric signal from a received acoustic wave.

The supporting member 420 may contain a metallic material having a high mechanical strength. The supporting member 420 according to this exemplary embodiment is a shell having a hemispherical shape configured to support the receiving element group 410 along a hemispherical arc on the inner surface of the hemispherical shaped shell. In this case, the receiving elements 411 to 414 having directional axes toward the center of the curvature of the hemispherical arc are arranged on the inner surface of the hemispherical shaped shell. By imaging an electric signal group output from those receiving elements 411 to 414, the quality of the resulting image at the center of the curvature is increased. The configuration of the supporting member 420 is not limited to a hemispherical shaped shell. The supporting member 420 may have any configuration if it can support the receiving element group 410 in accordance with the shape of the object 1000. The supporting member 420 may have a plurality of receiving elements within a plane or a curved surface called 1D array, 1.5D array, 1.75D array, or 2D array.

The supporting member 420 according to this exemplary embodiment functions as a container accumulating the acoustic matching liquid 1100 thereinside.

The receiving unit 400 may include an amplifier circuit configured to amplify time-series analog signals output from the receiving elements. The receiving unit 400 may also include an A/D converter configured to convert time-series analog signals output from the receiving elements to time-series digital signals. In this regard, the receiving unit 400 may include the signal data collecting unit 600, which can incorporate therein the amplifier circuit and the A/D converter (not shown).

### Driving Unit 500

The driving unit 500 moves the light irradiating unit 100 and the receiving unit 400. The driving unit 500 includes a non-illustrated motor such as a stepping motor configured to generate driving force, a driving mechanism configured to transmit the driving force, and a position sensor configured to detect positional information regarding the receiving unit 400. The driving mechanism may be a leading screw mechanism, a link mechanism, a gear mechanism, or a hydraulic mechanism. The position sensor may be an encoder or a potentiometer such as a variable resistor. The driving unit 500 can change the relative positions of the object 1000 and the receiving unit 400 with respect to each other. The driving unit 500 can change the relative positions of the object 1000 and the receiving unit 400 linearly one dimensionally, two-dimensionally, or three-dimensionally, and rotationally in a circular or elliptical manner.

The driving unit 500 may change the relative positions of at least one of the light irradiating unit 100 and receiving unit 400 and the object 1000. In other words, the driving unit 500 may only be required to move at least one of the light irradiating unit 100, the receiving unit 400, and the object 1000. In order to move the object 1000, the holding unit 1200 holding the object 1000 may be moved to move the object 1000. The driving unit 500 may move the relative positions serially or by performing a step-and-repeat operation.

### Signal Data Collecting Unit 600

The signal data collecting unit 600 includes an amplifier configured to amplify electric signals being analog signals output from the receiving elements 411 to 414 and an A/D converter configured to convert the analog signals output from the amplifier to digital signals. The digital signals output from the signal data collecting unit 600 are stored in a storage unit 710 within the computer 700. The signal data collecting unit 600 is also called a Data Acquisition System (DAS). The electric signals herein conceptually include an analog signal and a digital signal. The signal data collecting unit 600 is connected to a light detecting sensor attached to a light emitting unit in the light irradiating unit 100 and may start processing in synchronism with the pulsed light 130 emitted from the light irradiating unit 100 as a trigger.

### Computer 700

The computer 700 includes a storage unit 710, a signal selecting unit 720, a speed-of-sound acquiring unit 730, an object information acquiring unit 740, and a control unit 760. Functions of these components will be described in descriptions regarding the processing flow.

The storage unit 710 may be a non-transitory storage medium such as a ROM (Read only memory), a magnetic disk, and a flash memory. Alternatively, the storage unit 710 may be a volatile medium such as a RAM (Random Access Memory). A storage medium storing a program is non-transitory.

A unit responsible for an arithmetic function as a processing unit such as the signal selecting unit 720, the speed-of-sound acquiring unit 730, or the object information acquiring unit 740 may include a processor such as a CPU or a GPU (Graphics Processing Unit) and an arithmetic circuit such as an FPGA (Field Programmable Gate Array) chip. These units may include a single processor and a single arithmetic circuit but may include a plurality of processors and a plurality of arithmetic circuits instead.

The control unit 760 may include an arithmetic element such as a CPU. The control unit 760 controls over operations of the components of the photoacoustic apparatus. The control unit 760 may control over the components of the photoacoustic apparatus in response to an instruction signal through an operation, for example, to start a measurement from the input unit 900. The control unit 760 may read out program code stored in the storage unit 710 to control an operation of the corresponding component in the photoacoustic apparatus.

The computer 700 may be a specially designed workstation. The components of the computer 700 may be configured by hardware elements different from each other. At least partial components of the computer 700 may be configured by a single hardware element.

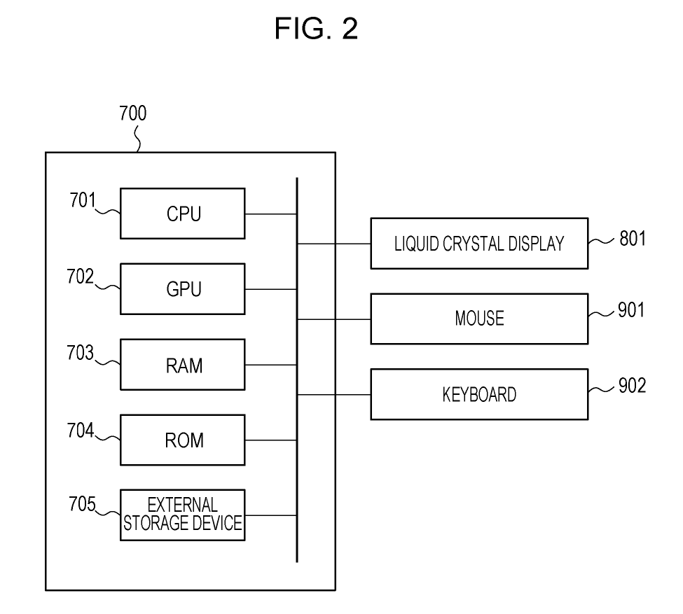

FIG. 2 illustrates a concrete configuration of the computer 700 according to this exemplary embodiment. The computer 700 according to this exemplary embodiment includes a CPU 701, a GPU 702, a RAM 703, a ROM 704, and an external storage device 705. To the computer 700, a liquid crystal display 801 functioning as the display unit 800, a mouse 901 functioning as the input unit 900, and a keyboard 902 are connected.

### Display Unit 800

The display unit 800 is a display device such as a liquid crystal display and an organic electro luminescence. The display unit 800 is a device configured to display an image based on object information acquired by the computer 700 and a numerical value associated with a specific position. The display unit 800 may display a GUI usable for manipulating an image or operating a device.

### Input Unit 900

The input unit 900 may include a mouse and a keyboard which are operable by a user. The display unit 800 may include a touch panel so that the display unit 800 can also function as the input unit 900.

The components of the photoacoustic apparatus may be provided as separate devices or may be provided as one apparatus in which they are integrated. At least partial components of the photoacoustic apparatus may be provided as a single integrated apparatus.

### Object 1000

Though the object 1000 is not a component of the photoacoustic but the object 1000 will be described below. The photoacoustic apparatus according to this exemplary embodiment may be used for diagnoses of a malignant tumor or a vascular disease in a human or an animal or for follow-up after a chemical treatment. Therefore, the object 1000 may be an objective region for a diagnosis such as a living body, more specifically, the breast, the neck, or the abdomen, for example, of a human or animal body. For example, in a case where a human body is to be measured, oxyhemoglobin or deoxyhemoglobin or a blood vessel containing them or a neovessel formed in vicinity of a tumor may be an optical absorber.

### Acoustic Matching Liquid 1100

Though the acoustic matching liquid 1100 is not a component of the photoacoustic apparatus, it will be described below. The acoustic matching liquid 1100 is usable for propagating acoustic waves between the holding unit 1200 and the receiving elements 411 to 414. The acoustic matching liquid 1100 may be water or ultrasound gel. The acoustic matching liquid 1100 may have less acoustic wave attenuation. In a case where irradiated light transmits through the acoustic matching liquid, the acoustic matching liquid may be transparent to the irradiated light. The acoustic matching liquid 1100 is filled between the object 1000 and the holding unit 1200.

According to this embodiment, the supporting member 420 may function as a container storing the acoustic matching liquid 1100. The object information acquiring apparatus may have a container capable of accumulating the acoustic matching liquid 1100 between the receiving elements 411 to 414 and the object 1000 in addition to the supporting member 420.

The acoustic matching liquid 1100 may be disposed between the object 1000 and the holding unit 1200. This embodiment assumes that the object 1000 and the holding unit 1200 are acoustically matched.

### Folding Unit 1200

The holding unit 1200 is usable for retaining the shape of an object during a measurement. By holding the object 1000 in the holding unit 1200, the object can be prevented from moving, and the position of the object 1000 can be held within the holding unit 1200. The holding unit 1200 may be made of a material such as PET-G.

The holding unit 1200 may be made of a material having a hardness capable of retaining the object 1000. The holding unit 1200 may be made of a material allowing light used for measurement to pass through. The holding unit 1200 may be made of a material having a substantially equal impedance to that of the object 1000. In a case where the object 1000 is one having a curved surface such as the breast, the holding unit 1200 may be concave-shaped. In this case, the object 1000 may be inserted to the concave part of the holding unit 1200.

The photoacoustic apparatus may not have the holding unit 1200 if the object 1000 is not required to be retained.

### Liquid Level Detecting Unit 1300

The liquid level detecting unit 1300 detects the position of the liquid level 1110 of the acoustic matching liquid 1100 and transmits information regarding the position (level) of the liquid within the container to the computer 700. The liquid level detecting unit 1300 may be a floating type sensor, electrode type sensor, optical, ultrasonic, capacitive, guide pulse, or pressure type liquid level sensor. The liquid level detecting unit 1300 is disposed at a position where reception of acoustic waves is not hindered (or a position at least off the directional axes of the converting elements).

### Supply Unit 1400

The supply unit 1400 may supply acoustic matching liquid to a space formed by the supporting member 420 functioning as a container in which the acoustic matching liquid can be placed. The supply unit 1400 has an accumulating unit configured to accumulate acoustic matching liquid and a pump feeding unit configured to pump the send the matching liquid from the accumulating unit to a space for acoustic matching liquid. The supply unit 1400 may further include a temperature control unit configured to control the temperature of acoustic matching liquid to be supplied, a valve configured to adjust the amount to be supplied, and a filter configured to remove impurities contained in the acoustic matching liquid. The temperature control unit has a function of measuring the temperature of acoustic matching liquid to be fed into the container, and a function of heating or cooling the acoustic matching liquid to a target temperature. Thus, fluctuations in temperature condition of the acoustic matching liquid and a component in contact with the acoustic matching liquid can be suppressed for stable measurement. The valve is usable for adjusting the flow rate and pressure of a lubricant. The filter functions for removing impurities such that the impurity mixed during circulation of the lubricant can be prevented from entering to the space formed by the container. For example, the filter may have a mesh structure. Thus, occurrence of measurement noise due to such impurities and a scratch due to such impurity can be prevented.

At least partial components of the photoacoustic apparatus as described above can be implemented by a single hardware apparatus. For example, the receiving unit 400 and the signal data collecting unit 600 may be enclosed in a common housing.

### Object Information Acquiring Method

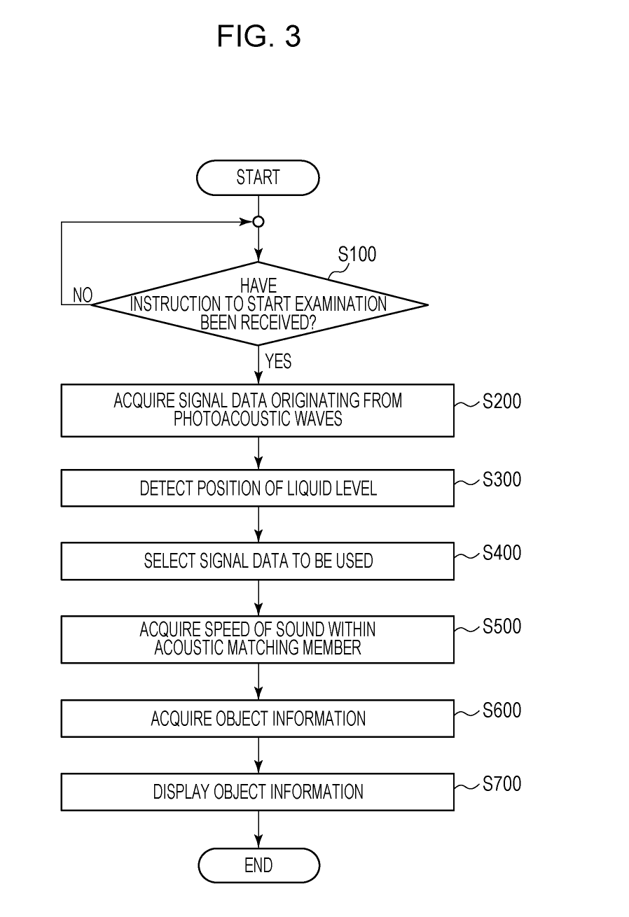

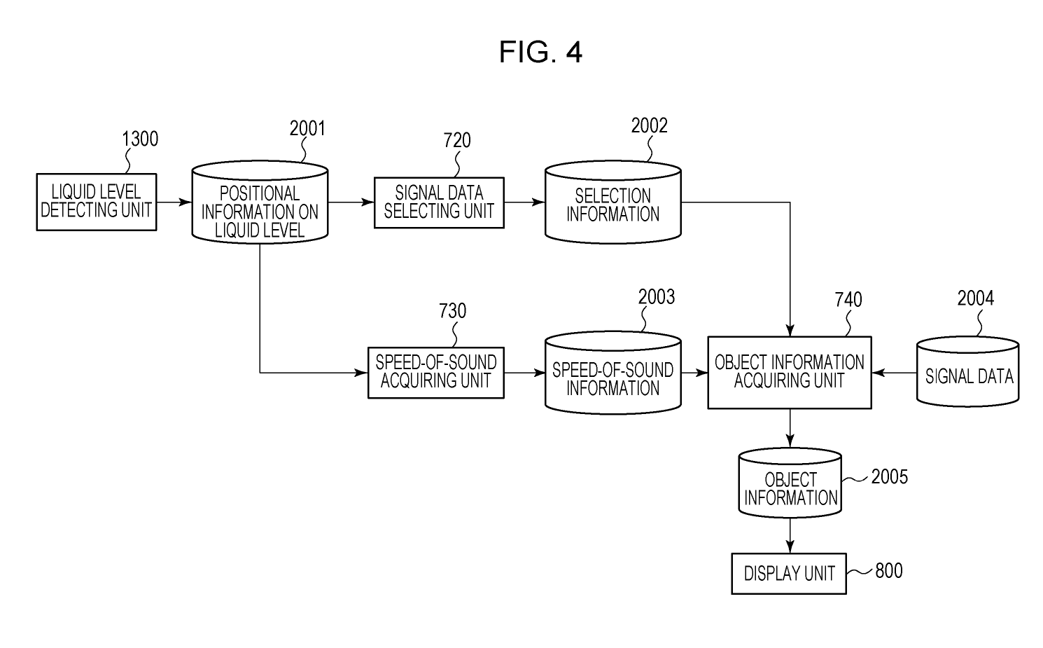

Next, an object information acquiring method using the photoacoustic apparatus according to this exemplary embodiment will be described. FIG. 3 is a processing flowchart illustrating the object information acquiring method according to this exemplary embodiment. FIG. 4 illustrates a dataflow of the object information acquiring method according to this exemplary embodiment.

S100: Step of Determining Whether an Instruction to Start a Measurement has been Received or not

The control unit 760 waits for an instruction to start a measurement from the input unit 900. In response to a signal regarding an instruction to start a measurement from the input unit 900, the control unit 760 transmits a control signal for starting a measurement to the corresponding components of the photoacoustic apparatus. If a user performs an operation input by for starting a measurement by using the input unit 900, the processing moves to S200.

S200: Step of Acquiring Signal Data Originating from Photoacoustic Waves

According to this exemplary embodiment, the driving unit 500 moves the optical system 120 and the supporting member 420 in synchronization, and the optical system 120 moves and at the same time irradiates pulsed light 130 to the object 1000. The receiving elements 411 to 414 also move and at the same time receive the photoacoustic waves.

The light source 110 generates the pulsed light 130 at a predetermined repeating frequency (such as 10 Hz). The driving unit 500 moves the optical system 120 so that the pulsed light 130 can be irradiated to different positions of the object 1000 at predetermined periods. In other words, the light irradiating unit 100 can irradiate the pulsed light 130 to the object 1000 at a plurality of time points.

The receiving elements 411 to 414 receive photoacoustic waves at different positions at every light irradiation time point and output electric signals corresponding to the irradiation time points. Such electric signals output at every light irradiation time point will collectively be called a plurality of electric signals corresponding to a plurality of light irradiation time points.

The signal data collecting unit 600 performs at least AD conversion processing on the electric signals being analog signals output from the receiving elements 411 to 414 and store them in the storage unit 710 as signal data 2004.

### S300: Step of Detecting a Position of a Liquid Level

The liquid level detecting unit 1300 detects a position of the liquid level 1110 of the acoustic matching liquid 1100 accumulated in the supporting member 420. The liquid level detecting unit 1300 transmits information 2001 regarding the position of the liquid level 1110 of the acoustic matching liquid 1100 to the computer 700.

The liquid level detecting unit 1300 may detect the position of the liquid level 1110 at a time point when acoustic waves are received by the receiving elements 411 to 414.

The liquid level detecting unit 1300 may detect the position of the liquid level 1110 of the acoustic matching liquid 1100 at a time point when the pulsed light 130 is irradiated to the object 1000. For example, the photoacoustic apparatus may include a beam splitter or an optical fiber functioning as a branch unit by which the pulsed light 130 generated from the light source 110 branches off and a light-sensitive detector such as a photodetector (PD) configured to detect branch light. The light-sensitive detector may be a photomultiplier tube (PMT) using a photoelectric effect, a photoconductive cell using changes in electric resistance due to light irradiation, or a photovoltaic photodiode using a semiconductor pn junction. The liquid level detecting unit 1300 acquires a time point when the pulsed light 130 is irradiated to the object 1000 based on an output from the light-sensitive detector and, in response to the output as a trigger, detects the position of the liquid level 1110. In other words, the liquid level detecting unit 1300 may detect the position of the liquid level periodically every emission of pulsed light. The liquid level detecting unit 1300 may detect the position of the liquid level at periods each equal to an integral multiple of the emission period of pulsed light and estimate, by interpolation processing, for example, the position of the liquid level at a time point when the detection is not performed. According to this exemplary embodiment, while the driving unit 500 is moving the supporting member 420 functioning as a container, the light irradiating unit 100 irradiates the pulsed light 130 to the object 1000 at a plurality of time points. The liquid level detecting unit 1300 detects the position of the liquid level 1110 for each light irradiation. Thus, even when the liquid level 1110 of the acoustic matching liquid 1100 changes because of a movement of the supporting member 420, the position of the liquid level 1110 when a photoacoustic wave is received can be detected in real time.

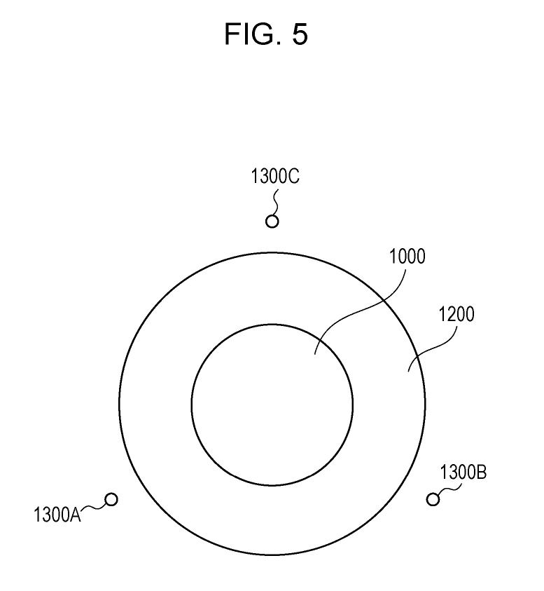

FIG. 5 is a top view of an exemplary layout of the liquid level detecting unit 1300 illustrated in FIG. 1. The liquid level detecting unit 1300 illustrated in FIG. 5 includes a liquid level sensor 1300A, a liquid level sensor 1300B, and a liquid level sensor 1300C, and each of the sensors detects a liquid level at its corresponding sensor position. The computer 700 can estimate the position of the liquid level 1110 by using information regarding the liquid levels acquired by those sensors. Having described the example in which a position of the liquid level is estimated based on detection results from three liquid level sensors in FIG. 5, four or more liquid level sensors may be provided to estimate a position of the liquid level based on the detection results therefrom.

### S400: Step of Selecting Signal Data to be Used for Acquiring Object Information

The signal selecting unit 720 uses the information 2001 regarding the position of the liquid level 1110 acquired by the liquid level detecting unit 1300 to select signal data to be used in S600 from a plurality of signal data sets corresponding to the plurality of light irradiation time points stored in the storage unit 710. The signal selecting unit 720 transmits selection information 2002 representing information based on which the signal data to be used in S600 can be determined to the object information acquiring unit 740.

If the position of the liquid level 1110 at a certain light irradiation time point is off a predetermined numerical range, the signal selecting unit 720 may determine all signal data acquired at the time point as signal data not to be used in S600. In other words, if the position of the liquid level 1110 at a certain light irradiation time point is within the predetermined numerical range, the signal selecting unit 720 may determine all signal data acquired at the time point as signal data to be used in S600.

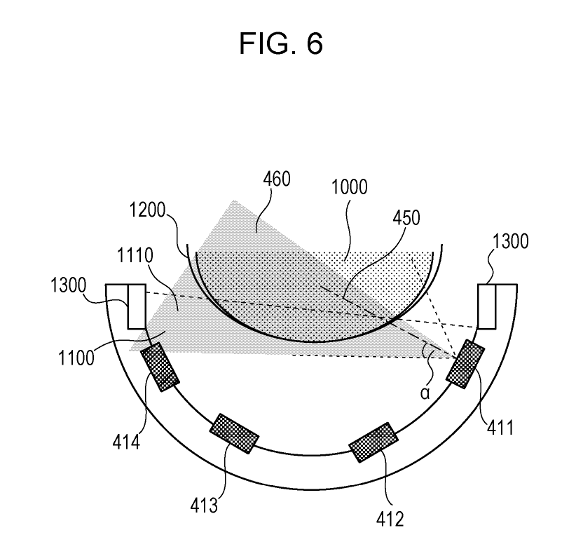

The signal selecting unit 720 being a determining unit may use information regarding the position of the liquid level 1110 to determine a receiving element which is not acoustically matched to the object 1000 and may not use, in S600, signal data originating from the electric signal output from the receiving element. If the position of the liquid level 1110 at a certain light irradiation time point is within the predetermined numerical range, the signal selecting unit 720 may select signal data to be used in S600 for each receiving element. Operations of the signal selecting unit 720 in a measurement state as illustrated in FIG. 6, for example, will be described. The signal selecting unit 720 uses information regarding a position of the liquid level 1110 to determine whether a surface of the object 1000 included in a range of a directivity angle α of the receiving element 411 is in contact with the acoustic matching liquid 1100 or not. If so, the signal selecting unit 720 may determine to use, in S600, the electric signal output from the receiving element 411. Referring to the case illustrated in FIG. 6, a part of a surface of the object 1000 included in a range corresponding to the directivity angle α of the receiving element 411 is not in contact with the acoustic matching liquid 1100. Thus, the signal selecting unit 720 may determine not to use, in S600, electric signals output from the receiving element 411. If a part of a surface of the object 1000 included in a range corresponding to the directivity angle α of the receiving element 411 is in contact with the acoustic matching liquid 1100, the signal selecting unit 720 may determine to use in S600 electric signals output from the receiving element 411. In other words, if it is determined that all of the surface of the object 1000 included in a range corresponding to the directivity angle α of the receiving element 411 is not in contact with the acoustic matching liquid 1100, the signal selecting unit 720 may determine not to use, in S600, electric signals output from the receiving element 411.

If the surface of the object 1000 in contact with a directional axis 450 (which is an axis in a direction with the highest receiving sensitivity) of the receiving element 411 is in contact with the acoustic matching liquid 1100, the signal selecting unit 720 may determines to use in S600 the electric signals output from the receiving element 411. In other words, the signal selecting unit 720 may determine not to use in S600 electric signals output from the receiving element 411 if the surface of the object 1000 in contact with the directional axis 450 of the receiving element 411 is not in contact with the acoustic matching liquid 1100. In this case, also if a part of the surface of the object 1000 included in a range corresponding to the directivity angle α of the receiving element 411 is in contact with the acoustic matching liquid 1100, the signal selecting unit 720 may determine not to use in S600 electric signals output from the receiving element 411.

Whether electric signals output from other receiving elements are to be used in S600 or not may be determined in the same manner. The same result as the determination result from the electric signals output from the receiving element 411 may be applied to a receiving element in vicinity of the receiving element 411. In other words, the determination result for a receiving element of a plurality of receiving elements may be handled as a determination result for a part of the remaining receiving elements.

According to this embodiment, whether signal data stored in the storage unit 710 are to be used for signal processing, which will be described below, based on information regarding a position of the liquid level of the acoustic matching liquid 1100. However, it may begin with determination on whether such signals are to be stored in the storage unit 710 or not.

In other words, the signal selecting unit 710 may use information regarding a position of the liquid level 1110 to identify a receiving element which is not acoustically matched to the object 1000, and the control unit 760 may controls the corresponding components not to store signals output from the receiving element in the storage unit 710. This prevents storage of non-accurate signals, reduces processing time by not performing storage of such signals, and saves storage capacity of the storage unit 710.

On the other hand, the signal selecting unit 720 may use information regarding a position of the liquid level 1110 to identify a receiving element which is acoustically matched to the object 1000, and the control unit 760 may control the corresponding components to selectively store signals output from the receiving elements in the storage unit 710. Therefore, optimizing the storage capacity of the storage unit 710.

Under this control, signals output from receiving elements which are not acoustically matched to the object 1000 can be controlled to be stored, but not to be used in the signal processing, which will be described below.

S500: Step of Acquiring a Speed of Sound Value within Acoustic Matching Liquid

The speed-of-sound acquiring unit 730 acquires a speed-of-sound value within the acoustic matching liquid 1100 by using the information 2001 regarding a position of the liquid level 1110 acquired by the liquid level detecting unit 1300. The speed-of-sound acquiring unit 730 transmits the speed-of-sound information 2003 acquired in this step to the object information acquiring unit 740.

The speed of sound within the acoustic matching liquid 1100 varies in accordance with hydraulic pressure generated by the acoustic matching liquid 1100. The following description assumes a case where water is the acoustic matching liquid 1100. In this case, hydraulic pressure P can be represented by the following expression.

P=1.11+1.02663×10⁻¹D+2.691×10⁻⁷D²−4.11×10⁻¹²D³

where D is a depth (m) from the liquid level 1110.

A speed-of-sound correction value Vp (m/s) with the hydraulic pressure P can be represented by the following expression.

V_P=1.60272×10⁻¹P+1.0268×10⁻⁵P²+3.5216×10⁻⁹P³−3.3603×10⁻¹²P⁴

In other words, the speed-of-sound acquiring unit 730 add Vp to the speed-of-sound value (statistic) of the acoustic matching liquid 1100 in atmospheric pressure so that the speed-of-sound value at a depth D from the liquid level 1110. However, an embodiment of the present invention is not limited to this method if a speed-of-sound value can be acquired by using a pressure value within the acoustic matching liquid 1100 in accordance with a relationship between the pressure and the speed-of-sound value. Thus, the speed-of-sound acquiring unit 730 can acquire the speed-of-sound value within the acoustic matching liquid 1100 with high precision by correcting the speed-of-sound value by using information regarding a position of the liquid level 1110.

The speed-of-sound acquiring unit 730 may correct the speed-of-sound value within the acoustic matching liquid 1100 by using other information regarding acoustic matching liquid 1100 in addition to the information regarding hydraulic pressure within the acoustic matching liquid. For example, the speed-of-sound acquiring unit 730 may use information regarding a temperature of the acoustic matching liquid 1100 in addition to the information regarding hydraulic pressure within the acoustic matching liquid to correct the speed-of-sound value within the acoustic matching liquid 1100.

The speed-of-sound acquiring unit 730 may use a pressure distribution within the acoustic matching liquid 1100 to acquire, as speed-of-sound information, a speed-of-sound distribution within the acoustic matching liquid 1100 (speed-of-sound values at a plurality of positions within the acoustic matching liquid 1100). Then, the propagation time of acoustic waves can be acquired with high accuracy by using speed-of-sound values at a plurality of positions within the acoustic matching liquid 1100 in a propagation path from a sound source to the corresponding receiving element.

The speed-of-sound acquiring unit 730 may acquire, as speed-of-sound information, a representative value of speeds of sound within the acoustic matching liquid 1100. For example, the speed-of-sound acquiring unit 730 may acquire, as a representative value of speeds of sound, a mean value of speed-of-sound values at positions within the acoustic matching liquid 1100. The propagation distance from a sound source to the corresponding receiving element may be divided by a representative value of speeds of sound to easily acquire the propagation time from the sound source to the receiving element.

### S600: Step for Acquiring Object Information

The object information acquiring unit 740 acquires information (object information 2005) regarding the object 1000 by using the signal data 2004 stored in the storage unit 710 and transmits it to the display unit 800.

The object information acquiring unit 740 further acquires the object information 2005 by selectively using signal data determined to be used by the signal selecting unit 720 among the signal data 2004 stored in the storage unit 710 by using the selection information 2002. Thus, object information can be acquired without using signal data originating from electric signals output from a receiving element not acoustically matched to the object 1000. Therefore, the object information can contain a reduced noise component.

The object information acquiring unit 740 may use information regarding the position of the liquid level 1110 to determine the projection position of signal data. The object information acquiring unit 740 may project, at the projection position, signal data corresponding to the position to acquire object information at the projection position.

For example, the object information acquiring unit 740 may only reconstruct an acoustically matched region included in the directivity angle α by using signals from the receiving elements having the acoustic matching liquid 1100 in contact with a part of a surface of the object 1000 included in the range of the directivity angle α. For example, referring to a case illustrated in FIG. 6, the object information acquiring unit 740 may reconstruct a shaded region 460 only. The object information acquiring unit 740 may only reconstruct the region corresponding to the object 1000 within the region 460. Thus, signals originating from photoacoustic waves from the acoustically matched region can be used efficiently.

The object information acquiring unit 740 may use the speed-of-sound information 2003 representing speed-of-sound values within the acoustic matching liquid 1100 acquired in S500 in addition to the signal data 2004 to acquire object information. Because the speed-of-sound information 2003 is a speed-of-sound value corrected by using information regarding the position of the liquid level 1110 as described above, the object information acquiring unit 740 can estimate the propagation time with high accuracy. Thus, the object information acquiring unit 740 may use information regarding the highly accurately estimated propagation time to estimate the position of the sound source with high accuracy. In other words, the object information acquiring unit 740 can acquire object information with high accuracy.

Pressure applied from the acoustic matching liquid to a receiving element may possibly change the level of the electric signals output from the receiving element. Accordingly, the object information acquiring unit may calculate the pressure applied from the acoustic matching liquid to a receiving element by using information regarding the position of the liquid level of the acoustic matching liquid. The object information acquiring unit may then correct the level of the electric signals (signal data) output from the receiving element by using information regarding the pressure applied from the acoustic matching liquid to the receiving element. Furthermore, the object information acquiring unit may acquire object information by using the signal data having the corrected level.

According to the present invention, the processing unit can apply any information processing method if electric signals are processed by using information regarding the position of the liquid level of the acoustic matching liquid. A plurality of processes or one process may be executed by using information regarding the position of the liquid level of the acoustic matching liquid.

### S700: Step for Displaying Object Information

The control unit 760 is configured to transmit the object information 2005 to the display unit 800 and cause the display unit 800 to display a numerical value and an image of object information. A doctor as a user can make a diagnosis by checking the numerical values and images of the object information displayed on the display unit 800.

Having described that, according to this exemplary embodiment, the photoacoustic apparatus is an object information acquiring apparatus, an ultrasonic diagnosis apparatus which acquires object information by exchanging ultrasonic waves may be applied as an object information acquiring apparatus according to the present invention. The object information acquired by the ultrasonic diagnosis apparatus may be a B mode image representing differences in acoustic impedance within an object (boundary information of tissue), information regarding Elastography within an object, blood flow information (doppler information) or the like. In this case, the ultrasonic diagnosis apparatus being the object information acquiring apparatus may have a transmitting unit configured to transmit transmission acoustic waves to an object. A receiving unit therein receives a reflected wave (echo) of a transmission acoustic wave and outputs an electric signal. The transmitting unit and the receiving unit may include a common receiving element or may include separate receiving elements. The liquid level detecting unit may detect the liquid level of the acoustic matching liquid at a time point when a transmission acoustic wave is transmitted to an object. The liquid level detecting unit may estimate a time point when a transmission acoustic wave is transmitted to an object based on a control signal regarding the transmission of the transmission acoustic wave output from the control unit to the transmitting unit.

The control unit 760 may control the components not to store an electric signal output from a receiving element determined as being not acoustically matched in S400 in the storage unit 710. In other words, the control unit 760 may select an electric signal to be stored in the storage unit 710 by using the selection information 2002. For example, the signal data collecting unit 600 may not perform AD conversion on an electric signal output from the receiving element so that the signal data can be stored in the storage unit 710. The signal data collecting unit 600 may perform AD conversion on an electric signal output from the receiving element and then may not store it in the storage unit 710. The signal data originating from an electric signal output from an acoustically matched receiving element and stored in the storage unit 710 may be used selectively to acquire object information. Thus, because object information can be acquired without using an electric signal output from a receiving element that is not acoustically matched, the object information may be prevented from being deteriorated.

### Second Exemplary Embodiment

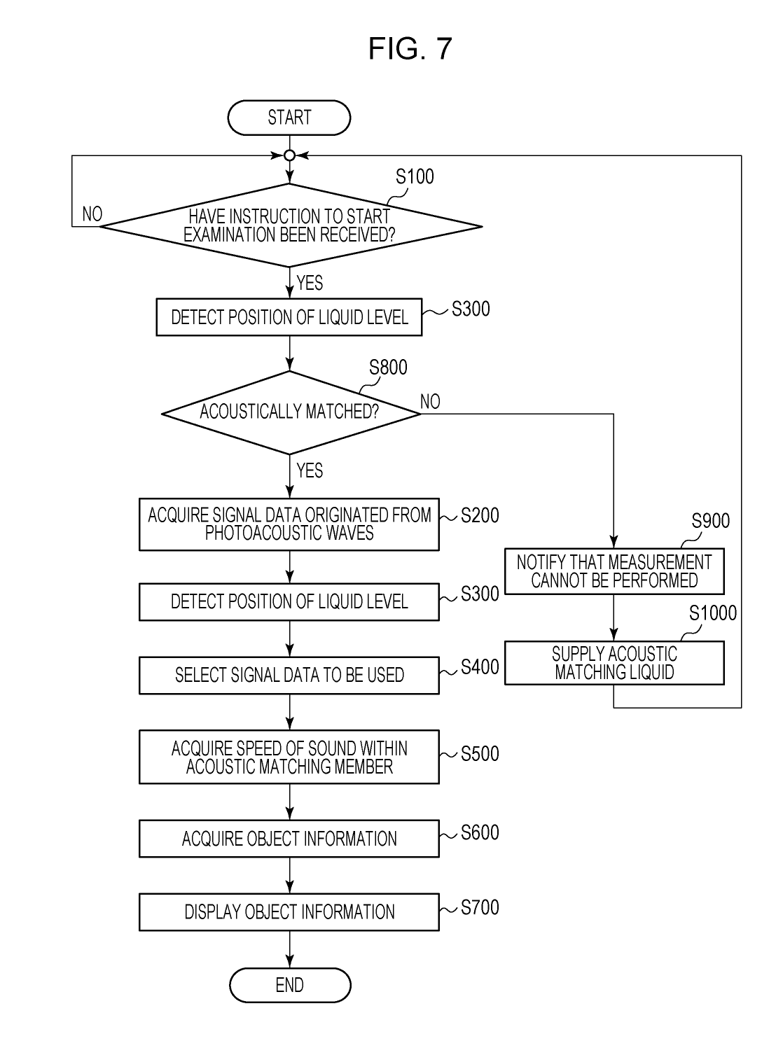

Next, a second exemplary embodiment will be described in which, before a photoacoustic measurement, the liquid level detecting unit 1300 detects a position of the liquid level 1110 of the acoustic matching liquid 1100 and determines whether a photoacoustic measurement can be performed or not. FIG. 7 illustrates processing flowchart of an object information acquiring method according to the second exemplary embodiment. Like numbers refer to like parts in FIG. 3 and FIG. 7, and any repetitive description will be omitted. This exemplary embodiment will be described with reference to the photoacoustic apparatus illustrated in FIG. 1.

### S800: Step for Determining Whether a Receiving Unit is Acoustically Matched or not

As an acoustic matching determining unit, a processing unit within the computer 700 uses information regarding a position of the liquid level of the acoustic matching liquid 1100 to determine whether any element 411 to 414 of the receiving unit 400 is acoustically matched with the object 1000 or not. If it is determined that the receiving unit 400 is acoustically matched to the object 1000, the processing moves to S200. The photoacoustic apparatus then acquires object information, like in the first exemplary embodiment by performing processing steps S300 to S700. On the other hand, if it is determined that the receiving unit 400 is not acoustically matched to the object 1000, the processing moves to S900.

In this processing, at step S800, the acoustic matching determining unit determines whether each receiving element is acoustically matched to the object 1000 or not. The acoustic matching determining unit determines that the receiving unit 400 is acoustically matched to the object 1000 if a predetermined or higher proportion of receiving elements is acoustically matched to the object 1000. For example, if a proportion equal to or higher than 50% of receiving elements included in the receiving unit 400 is acoustically matched, it may be determined that the receiving unit 400 is acoustically matched to the object 1000.

Alternatively, a plurality of receiving elements may be divided into a plurality of groups, and if there is a predetermined or higher proportion of acoustically matched receiving elements, the acoustic matching determining unit may determine that the receiving unit 400 is acoustically matched. In this case, neighboring receiving elements may be put into an identical group.

If all receiving elements are acoustically matched to the object 1000, the acoustic matching determining unit may determine that the receiving unit 400 is acoustically matched to the object 1000. In other words, the acoustic matching determining unit may determine that the receiving unit 400 is not acoustically matched to the object 1000 if at least partial receiving elements of a plurality of receiving elements are not acoustically matched.

S900: Step for Notifying that No Measurement can be Made

The control unit 760 notifies that no measurement can be made to a user by displaying that a photoacoustic measurement cannot be performed on the display unit 800 functioning as a notifying unit. For example, a warning text “measurement cannot be performed” may be displayed on the display unit 800, or an icon for instructing to start a measurement displayed on the display unit 800 may be disabled to indicate that no measurement can be made. An embodiment of the present invention is not limited to the method which notifies a determination result to a user by displaying it on the display unit 800, but audio may be output through a speaker to notify that no measurement can be made. The presence/absence of lighting up of an indicator light such as an LED lamp attached to the photoacoustic apparatus or a change of the color of such an indicator light may be used to notify that no measurement can be made. In this case, such a speaker or an indicator light functions as the notifying unit. Any method may be applied for the notification if a determination result can be notified to a user. Alternatively, the notifying unit in S200 may notify a user that the apparatus can perform a photoacoustic measurement.

### S1000: Step for Supplying Acoustic Matching Liquid

The supply unit 1400 supplies acoustic matching liquid to a space formed by the supporting member 420 functioning as a container for accumulating acoustic matching liquid. After the supply unit 1400 supplies a predetermined amount of acoustic matching liquid, the processing may move to S100. In other words, after the supply unit 1400 supplies a predetermined amount of acoustic matching liquid, the processing waits for an instruction to start a measurement from a user again. Alternatively, instead of supplying additional acoustic matching liquid, the control unit 760 may cause the driving unit 500 to move the receiving unit 400 with respect to the object 1000 until at least a proportion equal to or higher than 50% of receiving elements included in the receiving unit 400 is acoustically matched to the object 1000.

Processing in S300 may be executed in parallel with supply of acoustic matching liquid by the supply unit 1400 or movement caused by the driving unit 500. The control unit 760 may control the supply unit 1400 to stop the supply of acoustic matching liquid by the supply unit 1400 or control the driving unit 500 to stop movement of the receiving unit 400 when the receiving unit 400 and the object 1000 are acoustically matched.

Determining whether the acoustic matching is achieved or not before a photoacoustic measurement is executed allows a photoacoustic measurement to be started with the acoustic matching. This can prevent deterioration of object information acquired as a result of the photoacoustic measurement.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all modifications and equivalent structures and functions.

This application claims the benefit of Japanese Patent Application No. 2015-192212, filed Sep. 29, 2015, which is hereby incorporated by reference herein in its entirety.

What is claimed is:

1. An apparatus for acquiring object information, comprising:
at least one receiving unit configured to receive an acoustic wave occurring in an object and output a signal;
a liquid level acquiring unit configured to acquire information regarding a position of a liquid level of acoustic matching liquid provided between the object and the at least one receiving unit; and
a processing unit configured to acquire object information by processing the signal by using the information regarding the position of the liquid level.

2. The apparatus according to claim 1, wherein
the processing unit
acquires information regarding a speed of sound within the acoustic matching liquid by using the information regarding the position of the liquid level; and
acquires the object information by using the information regarding the speed of sound within the acoustic matching liquid and the signal.

3. The apparatus according to claim 2, wherein
the processing unit
acquires information regarding a hydraulic pressure within the acoustic matching liquid by using the information regarding the position of the liquid level; and
acquires information regarding a speed of sound within the acoustic matching liquid by using the information regarding the hydraulic pressure within the acoustic matching liquid.

4. The apparatus according to claim 1, wherein
the processing unit
acquires a corrected signal by correcting a level of the signal based on the information regarding the position of the liquid level; and
acquires the object information by using the corrected signal.

5. The apparatus according to claim 4, wherein
the processing unit
acquires information regarding a hydraulic pressure applied to the at least one receiving element by the acoustic matching liquid by using the information regarding the position of the liquid level; and
acquires the corrected signal by correcting the level of the signal based on the information regarding the hydraulic pressure applied to the at least one receiving element.

6. The apparatus according to claim 1, wherein
the at least one receiving element includes a plurality of converting elements disposed at positions different from each other, and the plurality of converting elements receive acoustic waves and output a plurality of signals; and
the processing unit
acquires information regarding hydraulic pressures applied to the plurality of converting elements by the acoustic matching liquid by using the information regarding the position of the liquid level;
acquires a plurality of corrected signals by correcting the levels of the plurality of signals based on the information regarding the hydraulic pressures applied to the plurality of converting elements; and
acquires the object information by using the plurality of corrected signals.

7. The apparatus according to claim 1, wherein
the processing unit
determines whether the at least one receiving element and the object are acoustically matched through the acoustic matching liquid by using the information regarding the position of the liquid level; and
does not use the signal for acquiring the object information, in a case where it is determined that the at least one receiving element is not acoustically matched.

8. The apparatus according to claim 1, wherein
the at least one receiving element includes a plurality of converting elements disposed at positions different from each other; and
the processing unit
determines whether the plurality of converting elements and the object are acoustically matched through the acoustic matching liquid by using the information regarding the position of the liquid level; and
acquires the object information without using a signal output from a converting being determined as not being acoustically matched to the object.

9. The apparatus according to claim 1, wherein
the processing unit
determines a projection position of the signal by using the information regarding the position of the liquid level; and
acquires the object information by projecting the signal to the projection position.

10. The apparatus according to claim 1, wherein
the acoustic wave is an acoustic wave generated from light irradiated to the object;
the liquid level acquiring unit acquires the information regarding the position of the liquid level of the acoustic matching liquid at a time point when the light is irradiated to the object; and
the processing unit acquires the object information by processing the signal by using the information regarding the position of the liquid level of the acoustic matching liquid at the time point when the light is irradiated to the object.

11. The apparatus according to claim 9, further comprising:
a light source configured to emit light;
a light detecting unit; and
a branch unit configured to branch light occurring from the light source to a first light ray and a second light ray,
wherein the at least one receiving element output the signal by receiving an acoustic wave generated from the first light ray irradiated to the object;
the light detecting unit detects the second light ray; and
the liquid level acquiring unit acquires the information regarding the position of the liquid level of the acoustic matching liquid at a time point when the light is irradiated to the object based on an output from the light detecting unit.

12. The apparatus according to claim 1, wherein
the acoustic wave is a reflected wave of a transmission acoustic wave transmitted to the object;
the liquid level acquiring unit acquires the information regarding the position of the liquid level of the acoustic matching liquid at a time point when the transmission acoustic wave is transmitted to the object; and
the processing unit acquires the object information by processing the signal by using the information regarding the position of the liquid level of the acoustic matching liquid at the time point when the transmission acoustic wave is transmitted to the object.

13. The apparatus according to claim 12, further comprising:
a control unit configured to output a control signal relating to transmission of the transmission acoustic wave; and
a transmitting unit configured to transmit the transmission acoustic wave to the object based on the control signal,
wherein the liquid level acquiring unit acquires the information regarding the position of the liquid level of the acoustic matching liquid at the time point when the transmission acoustic wave is transmitted to the object based on the control signal.

14. The apparatus according to claim 1, wherein
the liquid level acquiring unit acquires information regarding a position of the liquid level of the acoustic matching liquid at a time point when the at least one receiving element receives the acoustic wave.

15. The apparatus according to claim 1, further comprising:
a container configured to be capable of accumulating the acoustic matching liquid between the object and the at least one receiving element; and
a driving unit configured to move the container,
wherein the at least one receiving element is disposed in the container; and
the liquid level acquiring unit acquires information regarding a position of a liquid level of the acoustic matching liquid while the container is being moved.

16. The apparatus according to claim 1, further comprising
a storage unit configured to store the signal,
wherein the processing unit acquires the object information by processing the signal stored in the storage unit by using the information regarding the position of the liquid level.

17. A method of acquiring object information, comprising:
acquiring a signal originating from an acoustic wave occurring in an object;
acquiring information regarding a position of a liquid level of acoustic matching liquid; and
acquiring object information by processing the signal by using the information regarding the position of the liquid level.

18. An information processing method, comprising:
acquiring information regarding a position of a liquid level of acoustic matching liquid;
identifying a receiving unit acoustically matched to an object through the acoustic matching liquid and a receiving unit not acoustically matched to the object through the acoustic matching liquid by using the information regarding the position of the liquid level;
storing a signal output from the receiving unit acoustically matched to the object and not storing a signal output from the receiving unit not acoustically matched to the object based on the identification result of the identifying; and
obtaining information about the object based on the signal output from the receiving unit acoustically matched to the object.

19. A non-transitory computer readable storage medium that stores a program causing a computer to execute the information processing method according to claim 17.

20. A non-transitory computer readable storage medium that stores a program causing a computer to execute the information processing method according to claim 18.
